(19) United States
(12) Patent Application Publication
(10) Pub. No.: US 2023/0114825 A1
(43) Pub. Date: Apr. 13, 2023

(54) De novo designed protein homodimers containing tunable symmetric pockets

(71) Applicant: University of Washington, Seattle, WA (US)

(72) Inventors: David Baker, Seattle, WA (US); Derrick Hicks, Seattle, WA (US)

(21) Appl. No.: 17/938,752

(22) Filed: Oct. 7, 2022

Related U.S. Application Data
(60) Provisional application No. 63/255,355, filed on Oct. 13, 2021.

(51) Int. Cl. C07K 14/47; C12N 15/63

(52) U.S. Cl. CPC C07K 14/47; C12N 15/63; A61K 38/00

(57) ABSTRACT

Polypeptides including an amino acid sequence at least 50% identical to the amino acid sequence selected from the group consisting of SEQ ID NOS: 1-3297 are provided, which are capable of forming protein homodimers containing tunable symmetric pockets.

### CROSS REFERENCE

This application claims priority to U.S. Provisional Application Ser. No. 63/255,355 filed Oct. 13, 2021, incorporated by reference herein in its entirety.

### FEDERAL FUNDING STATEMENT

This invention was made with government support under Grant Nos. R01 AG063 845 and U19 AG065156, awarded by the National Institutes of Health. The government has certain rights in the invention.

### SEQUENCE LISTING STATEMENT

A computer readable form of the Sequence Listing is filed with this application by electronic submission and is incorporated into this application by reference in its entirety. The Sequence Listing is contained in the file created on Aug. 31, 2022 having the file name “21-1198-US.xml” and is 4,748 kb in size.

### BACKGROUND

Cyclic two fold (C2) symmetric molecules are common in biology and medicine, such as HIV protease inhibitors (Erickson and Kempf 1994), iron sulfur clusters (Bandyopadhyay, Chandramouli, and Johnson 2008), and the chlorophyll special pair found in photosynthetic reaction centers (Oie, Maggiora, and Christoffersen 2009). To bind such compounds, C2 symmetric protein homodimers are advantageous because each protein monomer can make identical interactions with an asymmetric unit of the small molecule. A large set of protein scaffolds with C2 symmetric binding pockets spanning a wide range of sizes and shapes that can be functionalized without compromising stability could provide new enzymes, therapeutics, and light harvesting proteins, but neither such sets nor methods to generate them currently exist.

### SUMMARY

In one aspect, the disclosure provides polypeptides comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS:1-3297, not including any functional domains added fused to the polypeptides (whether N-terminal, C-terminal, or internal), and wherein the 1, 2, 3, 4, or 5 N-terminal and/or C-terminal amino acid residues may be present or absent and when absent are not considered in determining the percent identity. In one embodiment, all residues are included when determining the percent identity relative to the reference polypeptide. In another embodiment, amino acid substitutions relative to the reference polypeptide are conservative amino acid substitutions. In a further embodiment, the polypeptide further comprises one or more functional domains.

In one embodiment, the disclosure provides homodimers of a polypeptide of the disclosure. In one embodiment, the homodimer comprises a ligand bound in a pocket of the homodimer. In another embodiment, the ligand comprises a C2 symmetric compound.

In one embodiment, the disclosure provides compositions comprising 2, 5, 10, 15, 25, 50, 100, 250, 500, 750, 1000, 1250, 1500, 1750, 2000, 2250, 2500, 2750, or more different polypeptides or homodimers of any embodiment of the disclosure.

The disclosure also provides nucleic acids encoding polypeptides of the disclosure, expression vector comprising the nucleic acids operatively linked to a suitable control sequence, host cells comprising a polypeptide, homodimer, nucleic acid, and/or expression vector of the disclosure, pharmaceutical compositions comprising a polypeptide, homodimer, nucleic acid, expression vector, and/or host cell of the disclosure together with a pharmaceutically acceptable carrier, and methods for using a polypeptide, homodimer, nucleic acid, expression vector, host cell, and/or pharmaceutical composition of the disclosure.

### DESCRIPTION OF THE FIGURES

FIG. 1. Design strategy. A. Schematic of our design pipeline from curved repeat protein (left) to symmetric homodimers (center) to future C2 symmetric ligand binders (right). Hypothetical C2 symmetric ligands are shown. B. Example of repeat proteins sampling different curvature. Proteins with desired helical parameters are shown in boxes. The helical symmetry axis of the proteins are aligned to the z-axis, and an xyz axis is depicted to show that we are looking directly down the z-axis. C. A histogram of helical curvature for 1 million backbones made with or without our biased sampling method. D. A single monomer (left) can be docked into various rigid body orientations to create homodimers (right) with diverse central cavities. This docking approach can create head to tail (light box) or head to head +tail to tail (dark box) homodimers. E. Examples of homodimers featuring a range of cavity sizes and shapes. Each dimer is based on a different monomeric curved repeat protein. The top row shows proteins looking down the central cavity, depicted as backbone ribbon representation for both chains and with surface mesh on chain B. The bottom row shows a side view slicing through the protein to illustrate the shape of the central cavity, depicted as surface representation. Head to tail homodimers are shown in a light gray box and head to head+tail to tail homodimers are shown in a dark gray box. The C2 symmetry axis of the protein homodimer is indicated.

FIG. 2. Biophysical characterization. Representative data for 6 successful designs and 1 failed design as assessed by SAXS. Left: Design names along with the helical parameters (rise, radius, and curvature) of the associated protein monomer. Second column; design models depicted as ribbon backbones. Third column; normalized UV absorbance (A280) obtained during SEC-MALS, followed by circular dichroism scans from 200-260 nm at 25° C., 95° C. and 25° C. post heating. Fourth column; predicted SAXS profiles overlaid on experimental SAXS data points for scattering vector (q, from 0 to 0.25) vs intensity (I).

FIG. 3. Schematic of a repeat protein with corresponding repeat helical axis. On top a repeat unit is shown along with a black dot representing the repeat unit center of mass. Below, the center of mass of four repeat units are plotted along their central helical axis. The helical parameters, rise, radius, and curvature, which we used to supplement the coarse grained scoring function are shown.

FIG. 4. Example repeat proteins sampling different helical rise. A protein with desired helical parameters is shown in the middle. The helical axis of all proteins are aligned to the z-axis, and the corresponding xyz coordinate frame is shown.

FIG. 5. Scatter plot of the number of designs having desired helical parameters with biased sampling or without biased sampling according to the length of helix 1 and helix 2. The size of each circle is proportional to the number of designs with desired parameters (left) plus being well packed (right). 1 million trajectories were attempted with biased sampling and without biased sampling. Sampling was spread out equally across all helix 1 by helix 2 combinations.

FIG. 6. Three-dimensional landscape of repeat protein space for rise, radius, and curvature. 1 million trajectories for repeat proteins generated using biased sampling (left) or unbiased sampling (right) plotted on a three-dimensional grid for rise, radius, and curvature. Two proteins are shown from each of the biased and unbiased trajectories along with their helical parameters. The two proteins on the left represent the type of curved repeat proteins we aimed to generate.

FIG. 7. Histogram of SS Degree Worst, which measures the minimum number of neighboring helices that each central helix in a repeat protein contacts. Data for models produced without our biased fragment assembly protocol are shown as are data for models produced with our biased fragment assembly protocol.

FIG. 8. Design workflow showing the number of designs at each step along with filtering methods used to pick the best designs for subsequent steps.

FIG. 9. Designs span a diverse range of sizes and shapes. Shown are 72 ordered designs depicted as ribbon backbones.

FIG. 10. The right side shows boxplots plus points for four pocket features, volume, dimension 1, dimension 2, and dimension 3 for the top one thousand designs along with all ordered scaffold designs. The pocket features were calculated on poly-alanine backbones to represent the maximum possible size of the pockets.

FIG. 11. Head-to-tail homodimers (left) compared to head-to-head+tail-to-tail homodimers (right). The top shows the proteins looking down the axis of symmetry, and below, the proteins are rotated 180° to show the side of the protein. Proteins are shown in backbone cartoon representation.

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2nd Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

As used herein, the singular forms “a”, “an” and “the” include plural referents unless the context clearly dictates otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

In all embodiments of polypeptides disclosed herein, any N-terminal methionine residues are optional (i.e.: the N-terminal methionine residue may be present or may be absent).

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

In a first aspect, the disclosure provides polypeptides comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS:1-3297, not including any functional domains added fused to the polypeptides (whether N-terminal, C-terminal, or internal), and wherein the 1, 2, 3, 4, or 5 N-terminal and/or C-terminal amino acid residues may be present or absent and when absent are not considered in determining the percent identity.

As described in the examples that follow, the polypeptides of the disclosure are capable of forming C2 symmetric homodimers with a variety of C2 symmetric cavities, and may be used, by way of non-limiting example, as members of polypeptide libraries for making binding proteins.

In one embodiments, the polypeptide comprises an amino acid sequence least 75% identical to the amino acid sequence selected from the group consisting of SEQ ID NOS:1-3297, not including any functional domains added fused to the polypeptides (whether N-terminal, C-terminal, or internal), and wherein the 1, 2, 3, 4, or 5 N-terminal and/or C-terminal amino acid residues may be present or absent and when absent are not considered in determining the percent identity. In another embodiment, the polypeptide comprises an amino acid sequence at least 90%, 91%, 92%, 93%, 94%, 95%, 96%, 97%, 98%, 99%, or 100% identical to the amino acid sequence selected from the group consisting of SEQ ID NOS:1-3297, not including any functional domains added fused to the polypeptides (whether N-terminal, C-terminal, or internal), and wherein the 1, 2, 3, 4, or 5 N-terminal and/or C-terminal amino acid residues may be present or absent and when absent are not considered in determining the percent identity. In one embodiment, the polypeptide comprises the amino acid sequence selected from the group consisting of SEQ ID NOS:1-3297, not including any functional domains added fused to the polypeptides (whether N-terminal, C-terminal, or internal), and wherein the 1, 2, 3, 4, or 5 N-terminal and/or C-terminal amino acid residues may be present or absent and when absent are not considered in determining the percent identity. In another embodiment, all residues are included when determining the percent identity relative to the reference polypeptide.

In one embodiment, amino acid substitutions relative to the reference polypeptide are conservative amino acid substitutions. As used herein, “conservative amino acid substitution” means a given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are known. Polypeptides comprising conservative amino acid substitutions can be tested in any one of the assays described herein to confirm that a desired activity, e.g. antigen-binding activity and specificity of a native or reference polypeptide is retained. Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into H is; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In one embodiment, the polypeptides further comprise one or more functional domains. Any suitable functional polypeptide domain may be used, including but not limited to a detectable domain (fluorescent protein, amino acid tag, etc.) and a targeting domain (cell penetrating peptide, antibody, etc.).

In another embodiment, the disclosure provides homodimers of the polypeptides if the invention. As described in the examples that follow, the polypeptides of the disclosure are capable of forming C2 symmetric homodimers with a variety of C2 symmetric cavities that are capable of binding a wide range of C2 symmetric compounds. In one embodiment, the homodimer comprises a ligand bound in a pocket/cavity of the homodimer. In one such embodiment, the ligand comprises a C2 symmetric compound.

In a further embodiment, the disclosure provides compositions comprising 2, 5, 10, 15, 25, 50, 100, 250, 500, 750, 1000, 1250, 1500, 1750, 2000, 2250, 2500, 2750, or more different polypeptides or homodimers of any embodiment of the disclosure. In these embodiments, the polypeptides and homodimers may be used, for example, to screen for compounds that bind one or more of the polypeptides or homodimers.

In another embodiment, the disclosure provides nucleic acids encoding the polypeptide of any preceding embodiment of the disclosure. The nucleic acid sequence may comprise single stranded or double stranded RNA (such as an mRNA) or DNA in genomic or cDNA form, or DNA-RNA hybrids, each of which may include chemically or biochemically modified, non-natural, or derivatized nucleotide bases. Such nucleic acid sequences may comprise additional sequences useful for promoting expression and/or purification of the encoded polypeptide, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the disclosure.

In a further embodiment, the disclosure provides expression vectors comprising a nucleic acid of the disclosure operatively linked to a suitable control sequence. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector, or any other suitable expression vector.

In one embodiment, the disclosure provides host cell comprising the polypeptide, homodimer, composition, nucleic acid, and/or expression vector of any embodiment herein. The host cells can be either prokaryotic or eukaryotic. The cells can be transiently or stably engineered to incorporate the nucleic acids or expression vector of the disclosure, using techniques including but not limited to bacterial transformations, calcium phosphate co-precipitation, electroporation, or liposome mediated-, DEAE dextran mediated-, polycationic mediated-, or viral mediated transfection.

In a further embodiment, the disclosure provides pharmaceutical composition comprising:

(a) the polypeptide, homodimer, composition, nucleic acid, expression vector, and/or host cell of any embodiment herein; and

(b) a pharmaceutically acceptable carrier.

The pharmaceutical composition may comprise in addition to the component of the disclosure (a) a lyoprotectant; (b) a surfactant; (c) a bulking agent; (d) a tonicity adjusting agent; (e) a stabilizer; (f) a preservative and/or (g) a buffer.

The disclosure also provides methods for using, the polypeptide, homodimer, composition, nucleic acid, expression vector, host cell, and/or pharmaceutical composition of any embodiment herein for any suitable use as disclosed herein, such as binding C2 symmetric compounds for diagnostic and therapeutic use, and other uses as disclosed in the examples that follow.

The disclosure also provides methods for designing polypeptides that form homodimers containing tunable symmetric pockets, as disclosed in the examples that follow.

### EXAMPLES

Function follows form in biology, and the binding of small molecules requires proteins with pockets that match the shape of the ligand. When designing proteins that might bind symmetric ligands, protein homooligomers with matching symmetry are advantageous as each protein subunit can make identical interactions with the ligand. Here we describe a general approach to generate a library of hyperstable C2 symmetric proteins with pockets of diverse size and shape. We first designed repeat proteins that sample a continuum of curvatures but maintain low helical rise, then docked these into C2 symmetric homodimers to generate an extensive range of C2 symmetric cavities. 101 designs were experimentally characterized, and of these, the geometry of 31 were confirmed by small angle X-ray scattering. These scaffolds provide a rich set of proteins for binding a wide range of C2 symmetric compounds.

Cyclic two fold (C2) symmetric molecules are common in biology and medicine, such as HIV protease inhibitors (Erickson and Kempf 1994), iron sulfur clusters (Bandyopadhyay, Chandramouli, and Johnson 2008), and the chlorophyll special pair found in photosynthetic reaction centers (Oie, Maggiora, and Christoffersen 2009). To bind such compounds, C2 symmetric protein homodimers are advantageous because each protein monomer can make identical interactions with an asymmetric unit of the small molecule. A large set of protein scaffolds with C2 symmetric binding pockets spanning a wide range of sizes and shapes that can be functionalized without compromising stability could provide new enzymes, therapeutics, and light harvesting proteins, but neither such sets nor methods to generate them currently exist.

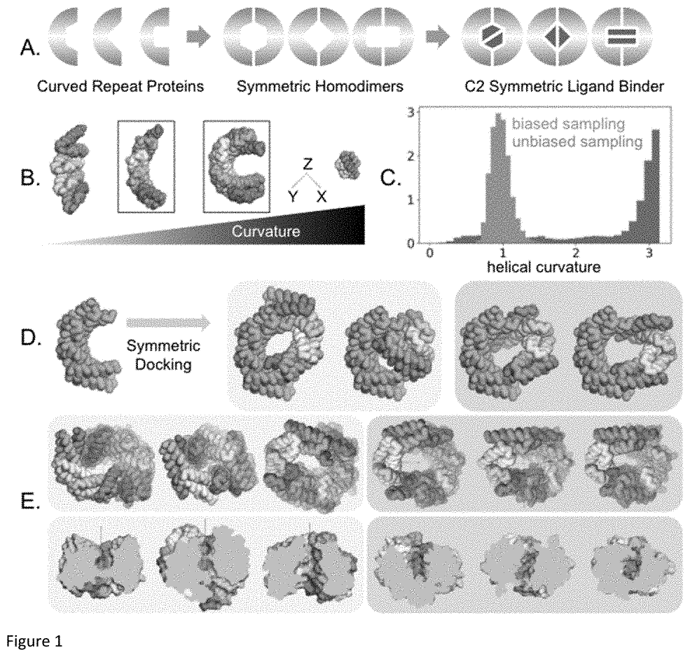

We set out to develop a general solution to the challenge of creating scaffold proteins for binding C2 symmetric ligands. We aimed to design repeat proteins that could house a central cavity, but with a wide range of elliptical, rather than perfectly circular, shapes to enable binding of a wider range of C2 symmetric ligands. We chose an overall design architecture consisting of repeat proteins which curve around a central axis that are docked into C2 symmetric homodimers surrounding an elliptical central cavity (FIG. 1A). By employing repeat proteins with minimal rise along the superhelical axis from one repeat unit to the next, we favor C2 arrangements with the ends of the two monomers in contact. Advantages of this conception are that the cavities can be vastly diverse in size, shape, and chemical composition (lined with different sidechain functional groups). Additionally, as the cavity lining residues are on the exterior of the monomers, the protein hydrophobic core is separated from the binding pocket; as such, functionalization to create binding interactions for specific compounds is unlikely to destabilize either the monomers or the dimer interface.

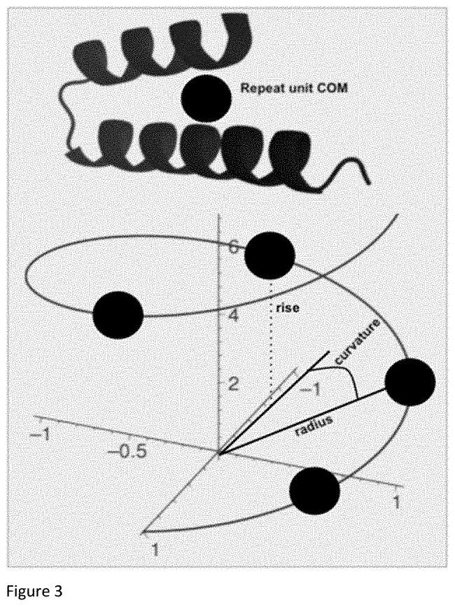

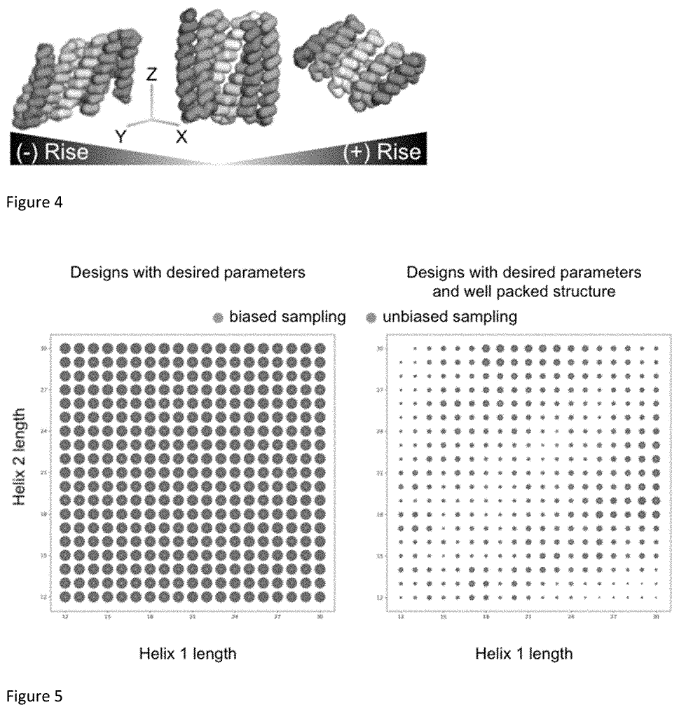

To implement this strategy to design C2 symmetric protein homodimers containing central cavities, we first set out to create a diverse library of monomeric units to dock into various symmetric homodimer orientations. We began by generating a new set of helical repeat protein monomers with structures specifically tailored for building C2 symmetric binding pockets. We selected a range of superhelical curvature, rise, and radius parameters, such that a four-unit repeat protein would approximate a half-circle and the resulting dimer would form an ellipse. Superhelical curvature and rise correspond to the rotation around and translation along the central superhelical axis per repeat unit respectively, and radius is the distance of the protein from this axis; these quantities are calculated from the center of mass of one repeat unit to the center of mass of the next repeat unit (see FIG. 3 for schematic) using the RepeatParameterFilter within the Rosetta' macromolecular modeling suite (Leman et al. 2020). Model building to approximate a half circle suggested the rotation between each repeat should be between 0.7 rad and 1.1 rad, the rise less than 1.5 A per repeat, and the radius between 10 Å and 22 Å. We hypothesized that, when docked into dimers, proteins with these parameters would create pockets that could accommodate ligands of diverse sizes and shapes. FIG. 1D/E and FIG. 4 illustrate how radius, curvature, and rise control the shape of repeat proteins and highlights the type of monomeric proteins we aimed to make.

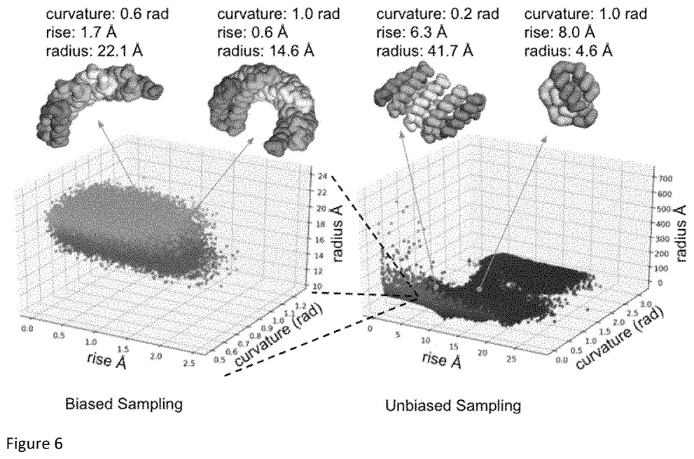

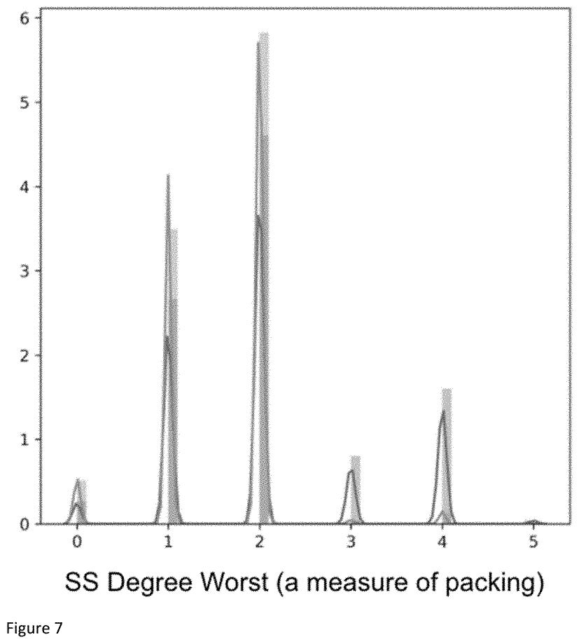

Unbiased sampling rarely yields repeat proteins with our desired helical parameters, and only in cases where the lengths of the two helices in a repeat differ by 6-7 residues (FIG. 5). We thus developed methods for biasing fragment assembly towards desired regions of repeat protein parameter space; at each fragment insertion (made identically in each repeat unit), the deviation from a target set of superhelical parameters is computed and the sum of these deviations is added to the coarse-grained score function previously used. With this biased assembly protocol, we were able to focus sampling on repeat protein structures with the desired superhelical parameters at all combinations of helix length (see FIG. 1C, FIG. 5, and FIG. 6); almost all trajectories with the biased fragment assembly protocol generate proteins with desired helical parameters (see FIG. 5). The new method led to poorly packed structures at a higher rate than the previous method (see FIG. 7), but after filtering these poorly packed structures out, we obtained far more curved repeat proteins at all helix length combinations using the new biased fragment assembly protocol (see FIG. 5). Obtaining a correct balance of scoring terms is critical, and these results suggest our helical parameter terms may be too strong, leading to structures that get stuck in poorly folded states that satisfy desired helical parameters. Despite this limitation, we were able to generate 100,000 curved repeat protein backbones to use for subsequent design.

These backbones were subjected to combinatorial sequence optimization using a Rosetta™ Scripts FastDesign protocol with repeat protein symmetry (applied through the RepeatProteinRelaxMover), which makes identical moves to each repeat unit during sequence design and minimization. The designs were then extended or shortened by up to half a repeat unit based on the energy per residue of the terminal helix to eliminate terminal helices that may be disordered due to limited contacts to the rest of the structure. The top 12,000 designs based on a combination of energy, packing, and sequence-structure agreement, were submitted for abinitio folding simulations. Designs for which the sequence strongly encoded the structure in de novo structure prediction calculations (see Methods; the lowest energy structures are close to the designed structure), 2,500 in total, were used in subsequent docking and design calculations.

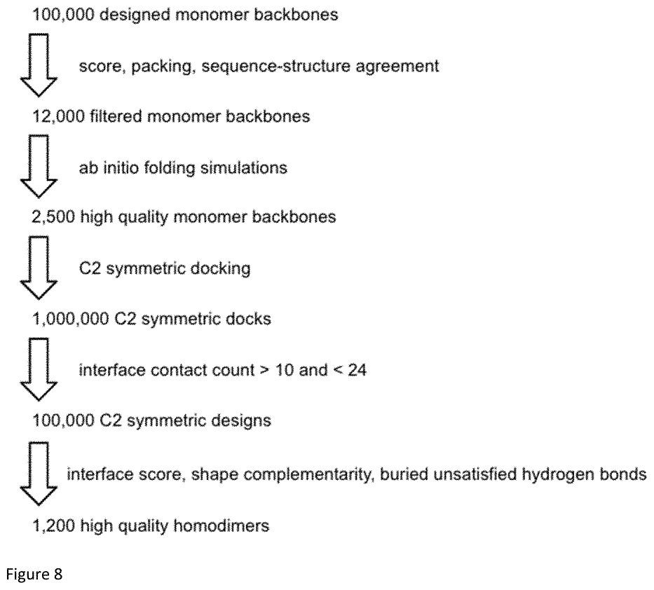

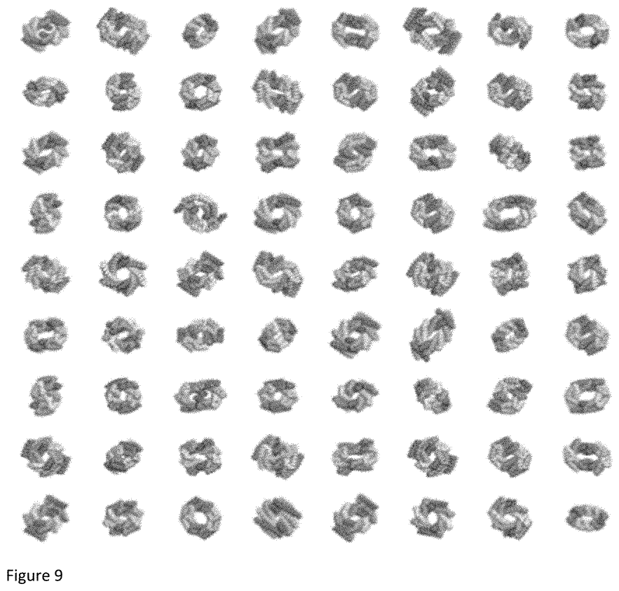

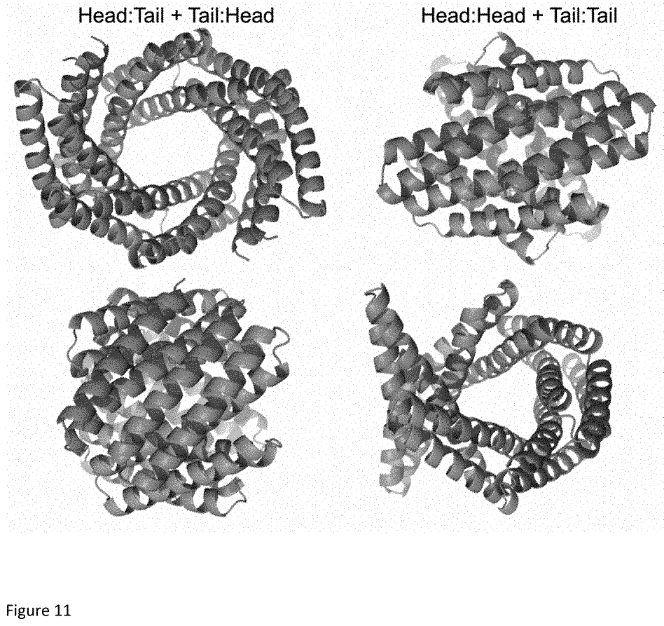

We next set out to create C2 symmetric homodimers with central cavities using these 2,500 curved repeat proteins. We extended a symmetric docking approach by adding a requirement that the docks create one of two classes of closed circular structure with either two N to C terminal interfaces (head to tail dimer) or both N to N and C to C terminal interfaces (head to head+tail to tail dimer) (See FIG. 11). This docking protocol generated 1 million docked structures. We subsequently removed docks that had small interfaces (less than 10 contacting residues) that were likely to form weak interfaces, as well as docks that had excessively large interfaces (greater than 24 contacts) which could lead to poor behavior before dimerization due to having many exposed hydrophobic residues. This yielded a set of about 100,000 docks for both classes of homodimers that were subjected to interface sequence optimization using a Rosetta™ Scripts FastDesign protocol (Maguire et al. 2021) with C2 symmetry (DiMaio et al. 2011). FIG. 1D shows how a single monomer can be docked into many distinct orientations creating diverse central cavities, and FIG. 1E shows examples of the diversity of proteins and pockets that can be achieved by docking diverse monomers into various C2 symmetric orientations. The top 1,200 homodimer designs were selected based on a combination of interface energy, interface shape complementarity, and buried unsatisfied hydrogen bonds for both classes of closed circular homodimers (see FIG. 8 for design flowchart).

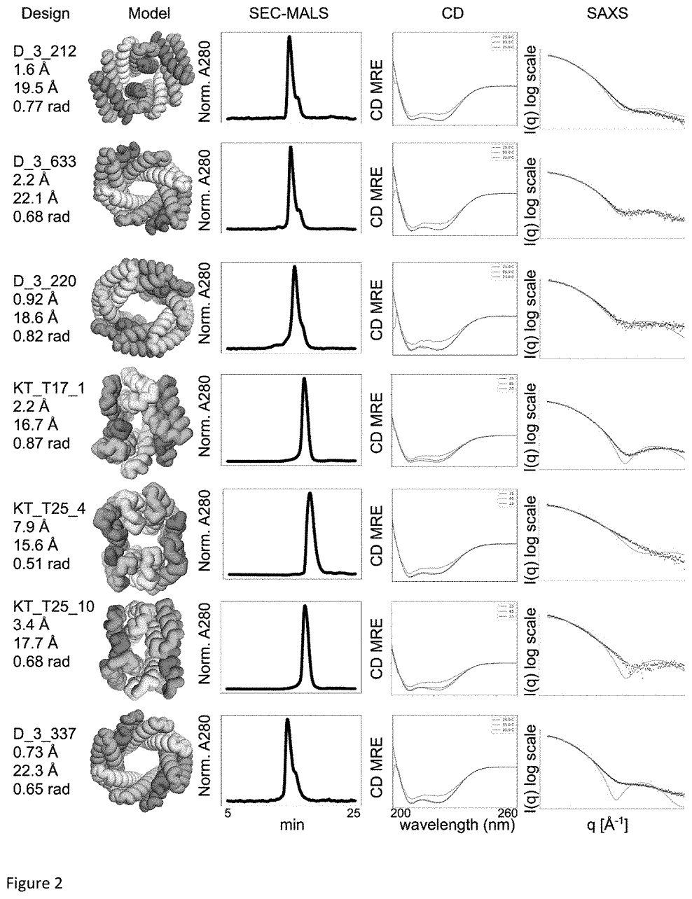

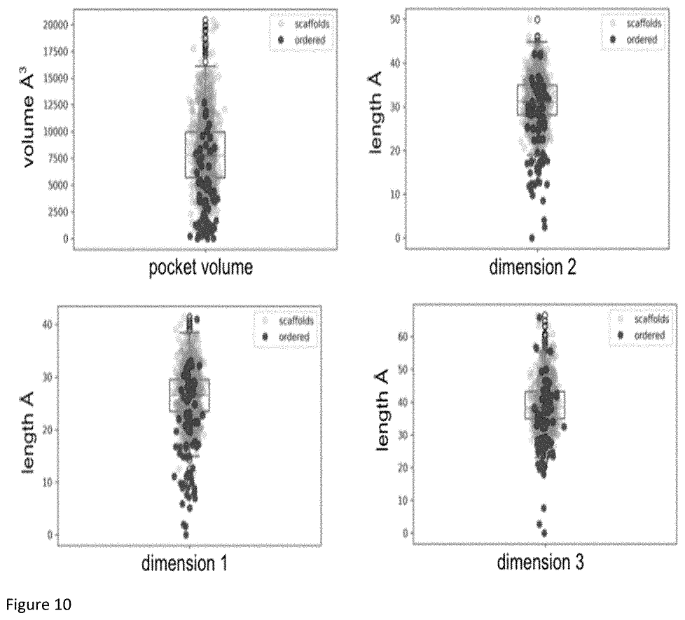

With the ability to generate these proteins computationally, we set out to characterize a diverse set of examples (see FIG. 9) with pockets varying in the volume and shape (see FIG. 10), approximated through the three principal axes, which were calculated on poly-alanine backbones to represent the maximum possible size of the pockets. FIG. 2 shows representative biochemical data for six successful homodimer designs and one design that failed to form the designed homodimer. In total, we characterized 101 designs including 77 head to tail dimers and 24 head to head+tail to tail dimers. 44 of the designs expressed enough soluble, well behaved, protein for further characterization. Five of these designs were determined to be soluble aggregate by subsequent analysis. Of the 39 remaining proteins, 38 were characterized by circular dichroism (CD), and of these, 36 were found to be helical and hyperstable (maintaining 80% helicity on average at 95° C.). All 36 of these proteins had nearly identical CD spectrums upon cooling back to 25° C. One design characterized by CD appeared helical as expected, but was not hyperstable, while another had low helical signal.

We subsequently used Small Angle X-ray Scattering (SAXS) to characterize the 37 designs that appeared helical by CD (including the one with low stability); of these, 31 had experimental SAXS profiles that closely matched profiles predicted for the corresponding design model, suggesting that they have the correct ellipsoidal shape in solution. In total, 31% of designs (31 of 101) are well-expressing soluble dimers that show experimental scattering profiles that closely match predicted profiles based on their design models. The failed design, D_3_337 is interesting, because it is hyperstable and helical by CD, but its SAXS data suggests it dimerizes to a different shape than designed. Designed proteins are disclosed in Tables 1-2 as SEQ ID NOS:1-3297

### Discussion

The C2 symmetric homodimer proteins created in this study have central cavities with diverse shapes that could accommodate a range of C2 symmetric ligands. These proteins have high thermal stability and solubility in a range of buffer conditions; some designs were concentrated over 100 mg/ml and remained soluble in crystal screens indefinitely. Because the protein core is distinct from the pocket, they should have high mutation tolerance during functionalization. The methods described here enable focused sampling of repeat protein conformational space beyond perfectly closing toroid structures; we used these methods to create a library of curved repeat proteins, but they could also be used to create a library of perfectly flat repeat proteins or to match the helical parameters of DNA or other helical biomolecules.

The dimeric ellipsoidal architecture of the proteins created here leads to a wide range of pocket sizes and geometries. A similar approach could be applied to the generation of higher order symmetric complexes to create pockets suitable for binding higher order symmetric molecules. Furthermore, many of the head to tail C2 symmetric designs described here could be connected into single chain proteins with short structured loops or long flexible linkers enabling them to be redesigned to bind arbitrary asymmetric small molecules or host enzyme active sites. The number of design models presented here already rivals the size of common fold classes found in the Protein Data Bank, and there is nearly unlimited ability to create more. Docking C2 symmetric compounds into these scaffolds can be carried out efficiently by superimposing the symmetry axes of the small molecule and protein scaffold, sampling the two remaining rigid body degrees of freedom (the translation along and rotation around the symmetry axes), and, for each dock, designing the protein interface to maximize interactions with the ligand.

### Methods

### Synthetic Gene Constructs

All genes were ordered from Integrated DNA Technologies (IDT). In a few cases, genes were not synthesizable by IDT, and were instead ordered from Genscript. A His-tag containing TEV protease cleavage site and short linkers were added to the N-terminus of protein sequences.

In cases in which the protein lacked a Tryptophan residue, a single Tryptophan was added to the short N-terminal linker following the TEV protease cleavage site to help with protein concentration quantification by A280. The protein sequence along with linker (GHHHHHHGSGSGENLYFQSGSGSSS (SEQ ID NO: 3298) or GHHHHHHGSGSGENLYFQSGWSGSSS (SEQ ID NO: 3299)) was reverse translated into DNA using a custom python script that attempts to maximize host-specific codon adaptation index(Sharp and Li 1987) and IDT synthesize-ability, which includes optimizing whole gene and local GC content as well as removing repetitive sequences. Finally, a TAA stop codon was appended to the end of each gene. Genes were delivered cloned into pET-29b+between NdeI/XhoI restriction sites.

### Protein Expression and Purification

Proteins were transformed into Lemo21(DE3) E. coli from New England Biolabs (NEB) and then expressed as 0.5-liter cultures in 2-liter flasks using Studiers M2 autoinduction media with 50 ug/mL kanamycin. The cultures were either grown at 37° C. for ˜6-8 hours and then ˜18° C. overnight (˜14 hours) or at 37° C. the entire time ˜14 hours. Cells were pelleted at 4,000 g for 10 minutes, after which the supernatant was discarded. Pellets were resuspended in 30 ml lysis buffer (25 mM Tris HCl pH 8, 300 mM NaCl, 30 mM imidazole, 1 mM PMSF, 0.75% CHAPS, 1 mM DNase, 10 mM Lysozyme, with Thermo Scientific Pierce protease inhibitor tablet). Cell suspensions were lysed by microfluidizer or sonication, and the lysate was clarified at 20,000g for ˜30 minutes. The His-tagged proteins were bound to Ni-NTA resin (Qiagen) during gravity flow and washed with a wash buffer (25 mM Tris HCl pH 8, 300 mM NaCl, 30 mM imidazole). Protein was eluted with an elution buffer (25 mM Tris HCl pH 8, 300 mM NaCl, 300 mM imidazole). The His-tag was removed by TEV cleavage, followed by IMAC purification to remove TEV protease. The flowthrough was collected and concentrated prior to further purification by SEC/FPLC on a Superdex™ 200 increase 10/300 GL column in TBS (25 mM Tris pH 8.0, 300 mM NaCl).

### Circular Dichroism

Circular dichroism spectra were measured with an AVIV Model 420 DC or Jasco J-1500 CD spectrometer. Samples were 0.25 mg/mL in TBS (25 mM Tris pH 8.0, 150 mM NaCl), and a 1-mm path length cuvette was used. The CD signal was converted to mean residue ellipticity by dividing the raw spectra by N×C×L×10, where N is the number of residues, C is the concentration of protein, and L is the path length (0.1 cm).

### Size Exclusion Chromatography with Multi-Angle Light Scattering

Purified samples after the initial SEC run, samples were pooled then concentrated or diluted as needed to a final concentration of 2 mg/mL. 100 uL of each sample was then run through a high-performance liquid chromatography system (Agilent) using a Superdex™ 200 10/300 GL column. These fractionation runs were coupled to a multi-angle light scattering detector (Wyatt) in order to determine the absolute molecular weights for each designed protein as described previously (Fallas et al. 2017).

### Small Angle X-Ray Scattering

Small-Angle X-ray Scattering (SAXS) was collected at the SIBYLS High Throughput SAXS Advanced Light Source in Berkeley, Calif. (Dyer et al. 2014). Beam exposures of 0.3 s for 10.2 s resulted in 33 frames per sample. Data was collected at low (˜1 mg/mL) and high (˜2-3 mg/mL) protein concentrations in SAXS buffer (25 mM Tris pH 8.0, 150 mM NaCl, 2% glycerol). The Sibyls™ website was used to analyze the data for high and low centration samples and average the best dataset. If there was obvious aggregation over the 33 frames, only the data points before aggregation arose were used in the Gunier region, otherwise, all data was included for the Gunier region. All data was used for Porod and Wide regions. The averaged file was used with scatter.jar to remove data points with outlier residuals in the Gunier region. Finally, the data was truncated at 0.25 q. This dataset was then compared to the predicted SAXS profile based on the design model using the FoxS SAXS server, and volatility ratio (Vr) was calculated to quantify how well the predicted and data matched the experimental data. Proteins with Vr of less than 2.5 were considered to be folded to the designed quaternary shape.

### Backbone Generation of Curved Repeat Protein Monomers

We generate repeat protein backbones using Rosetta™ Remodel which takes a blueprint file as input and performs Monte Carlo fragment assembly using a coarse-grained energy function to accept or reject moves (made identically in each repeat). A blueprint file describes a fixed-length protein by its secondary structure assignment at each residue position. The repeating unit of the repeat proteins we generate includes helix 1, loop 1, helix 2, and loop 2. We limit our search to short “ideal” loops, having a length of 2 to 4 residues, while helix lengths range from 12 to 30 residues. Within these limits, we enumerate all possible secondary structure length combinations and output a unique blueprint file for each combination. A Rosetta™ Scripts XML is used to run Rosetta™ Remodel on each blueprint to create four repeat unit proteins. Remodel begins by picking 3mer and 9mer fragments for each position based on the assigned secondary structure and then performs fragment assembly with these fragments. Trajectories begin with ideal helices at helical positions and extended loops. The protocol first makes fragment insertions at loop regions in order to quickly fold the largely extended protein chain into a globular protein, before performing fragment assembly over the full length of the protein. For this work, we ran 1 million total trajectories evenly distributed over all allowable secondary structure combinations (3249 total combinations), which yielded ˜300 models per secondary structure combination. We first did this with a traditional coarse-grained energy function supplemented with higher resolution, backbone only, residue pair motifs harvested from the PDB which generate better packed and designable protein models (Brunette et al. 2020; Fallas et al. 2017).

The aforementioned protocol failed to generate our desired shape of repeat proteins, specifically curved repeat proteins with low helical rise (<1.5 Å per repeat unit) and significant helical curvature (between 0.7 rad and 1.1 rad) that approximated a half-circle. To overcome this limitation and develop a method to focus sampling towards arbitrary repeat protein shapes, we developed three new score terms based on the helical parameters rise, radius, and curvature (often called omega or twist). At every fragment assembly step during the Monte Carlo protocol, the helical parameters of the protein are calculated as described elsewhere (Hauser et al. 2017) and then the deviation from a specified set of helical parameters is computed, and the sum of these deviations is added to the coarse-grained score function used earlier. The weight of these score terms is increased over the course of the trajectory. Furthermore, the score terms allow the user to use a linear or quadratic penalty based on the deviation or to set the penalty to 0 before or after the desired value is reached. The ability to turn off the penalty is important if the user wants to achieve a rise=0, in which case radius approaches infinity, which will cause scoring problems to arise for the radius term. In our study, we set the penalties to quadratic. Small scale testing showed this was appropriate, so we moved to larger-scale sampling identical to that described in the preceding paragraph, which generated an abundance of repeat protein with our desired helical parameters. We note that many trajectories got stuck in extended or spaghetti-like models that satisfied our desired helical parameters well at the expense of the other score terms.

### Sequence Design and Selection of Curved Repeat Proteins

The fragment assembly methods used to generate backbones will often create loops that are quite different (>0.4 Å rmsd) than those found in nature by recombining fragments in novel ways, which has been found to lower folding accuracy in subsequent in silico forward folding simulations (Brunette et al. 2020) and potentially during in vitro experiments too. To overcome this problem, every backbone is first subjected to a protocol that optimizes loops by replacing both unique loops in the repeat unit with all combinations of loops found in the PDB with <0.4 Å rmsd and then propagating these changes to each of the other repeat units (Brunette et al. 2020). This often results in input monomers creating several output monomers, although some inputs fail to find any loops with rmsd <0.4 Å and are discarded at this step.

After loop optimization, backbones are subjected to sequence optimization using a Rosetta™ Scripts XML. First, explicit repeat protein symmetry is applied to the computational model such that all subsequent steps of sequence mutation and backbone minimization will be done identically at each repeat position in the protein. The application of symmetry also reduces the design space to that of a single repeat unit plus its interface with neighboring units regardless of the number of repeat units (always 4 units in this work), which reduces the subsequent design time ˜4 fold. Next, PSSM generated from the sequence of 9-mer fragments found in the PDB and having low rmsd to the design model is applied to the protein to bias the score function towards the design of structurally appropriate amino acids. This 9-mer based structural PSSM is particularly helpful in the design of loop residues, i.e., in placing prolines and glycines in highly favorable positions where either inaccuracy in the Rosetta™ energy function would favor other residues or the lowest energy residue is not the best residue because of negative design considerations that exist outside absolute energetics. Similarly, this PSSM also helps in placing appropriate helix capping motifs such as aspartate, asparagine, serine, or threonine. Finally, the score function is modified to include an explicit penalty for buried unsatisfied hydrogen bonds (Coventry and Baker 2020).

FastDesign™ is then performed, which alternates between sequence optimization (amino acid mutations and rotamer exchanges) and backbone minimization through four cycles during which the weight of the repulsive energy term is ramped from low to high. LayerDesign is used during design to make sure the protein surface positions only mutate to polar residues. The protein core layer definition used allows mutations to small polar residues (D, H, N, Q, S, and T), but the buried unsatisfied penalty either prevents these entirely or forces them into fully satisfied polar networks which are sometimes desirable (Boyken et al. 2016). Next FastRelax is run which alternates between rotamer packing and backbone minimization similar to FastDesign. These two steps are then repeated, after which various filter metrics and scores are calculated to quantify the protein energetics, core packing, secondary structure shape complementarity, sequence-structure agreement, and buried unsatisfied hydrogen bonds.

Top designs based on protein energetics, core packing, secondary structure shape complementarity, sequence-structure agreement, and buried unsatisfied hydrogen bonds were subjected to forward folding simulations. Folding funnels with a forward folding metric, the area to the left of the folding funnel from the lowest energy point to +8 rosetta energy units, of less than 25 were chosen for subsequent design steps.

After forward folding, the N-terminus and C-terminus of the proteins were optimized by replacing terminal helices making minimal contacts to the rest of the protein with better packing termini. This was done by allowing the termini to extend or shorten by up to half a repeat unit to optimize the score per residue of the protein. We attempted to maintain the protein length as close to four repeat units as possible but favored lengthening the protein by half a repeat unit over shortening the protein. In the case that both termini were better when extended by half a repeat unit, which is functionally similar to truncating by half a repeat unit, we would make two combinations of the protein, first, a protein with the N-terminus extended and C-terminus truncated and second, a protein with the N-terminus truncated and C-terminus extended.

Finally, the whole protein surface was allowed to redesign for three reasons. The first was to break up the repetitive nature of the protein sequence, which makes DNA synthesis easier. The second was to allow better electrostatic complimentary on the surface because repulsive charges were sometimes forced next to each other due to the explicit repeat symmetry used to initially design the monomers. The third was to remove surface-exposed hydrophobic residues.

### C2 Symmetric Docking

We adapted a previous symmetric docking approach (Fallas et al. 2017) by adding a requirement that the N and C terminal helices of the monomers contact (at least one pair of residues on each terminus within 14 Å) in the dimer; this leads to head to tail homodimers with a closed circular structure and a central cavity along the axis of symmetry. Subsequently, we removed docks with small interfaces (less than 10 contacting residues) and excessively large interfaces (greater than 24 contacts).

### C2 Symmetric Protein-Protein Interface Design

Interface design was conducted by running a Rosetta™ Scripts XML that is highly similar to the one used to design monomers. The critical difference being that only interface residues are allowed to mutate during interface design. Interface residues are defined as residues with Ca atoms in one protein chain being within 10 Å of Ca atoms in the other protein chain. Additionally, C2 symmetry is applied during interface design, which serves a similar function to the repeat protein symmetry used during monomer design. C2 symmetry forces chain 1 and chain 2 to maintain identical sequences (and rotamers) during sequence optimization and identical torsion angles during minimization.

A similar trajectory of FastDesign, FastRelax, FastDesign, FastRelax is run as during monomer design. Loops, as defined by DSSP, are prevented from designing. Additionally, glycine and proline residues are not allowed to mutate, and no other positions can mutate to glycine or proline. Finally, we use an amino acid composition score term along with an explicit penalty for buried unsatisfied hydrogen bonds in order to favor the creation of small fully satisfied polar networks at the interface. This is done in the hopes of preventing the creation of large hydrophobic interfaces that might be prone to aggregation and to increase binding specificity of the interfaces. Finally, filter metrics and scores were calculated to quantify the binding energy, interface packing, interface shape complementarity, interface SASA, and buried unsatisfied hydrogen bonds across the interface.

Lengthy table referenced here
US20230114825A1-20230413-T00001
Please refer to the end of the specification for access instructions.

Lengthy table referenced here
US20230114825A1-20230413-T00002
Please refer to the end of the specification for access instructions.

### REFERENCES

An, Linna, and Gyu Rie Lee. 2020. “De Novo Protein Design Using the Blueprint Builder in Rosetta.” Current Protocols in Protein Science/Editorial Board, John E. Coligan . . . [et Al.] 102 (1): e116.
Bandyopadhyay, Sibali, Kala Chandramouli, and Michael K. Johnson. 2008. “Iron-sulfur Cluster Biosynthesis.” Biochemical Society Transactions. doi.org/10.1042/bst0361112.
Boyken, Scott E., Zibo Chen, Benjamin Groves, Robert A. Langan, Gustav Oberdorfer, Alex Ford, Jason M. Gilmore, et al. 2016. “De Novo Design of Protein Homo-Oligomers with Modular Hydrogen-Bond Network-Mediated Specificity.” Science 352 (6286): 680-87.
Brunette, T. J., Matthew J. Bick, Jesse M. Hansen, Cameron M. Chow, Justin M. Kollman, and David Baker. 2020. “Modular Repeat Protein Sculpting Using Rigid Helical Junctions.” Proceedings of the National Academy of Sciences of the United States of America 117 (16): 8870-75.
Brunette, T. J., Fabio Parmeggiani, Po-Ssu Huang, Gira Bhabha, Damian C. Ekiert, Susan E. Tsutakawa, Greg L. Hura, John A. Tainer, and David Baker. 2015. “Exploring the Repeat Protein Universe through Computational Protein Design.” Nature 528 (7583): 580-84.
Cohen-Ofri, Ilit, Maurice van Gastel, Joanna Grzyb, Alexander Brandis, Iddo Pinkas, Wolfgang Lubitz, and Dror Noy. 2011. “Zinc-Bacteriochlorophyllide Dimers in de Novo Designed Four-Helix Bundle Proteins. A Model System for Natural Light Energy Harvesting and Dissipation.” Journal of the American Chemical Society. doi.org/10.1021/ja202054m.
Coventry, Brian, and David Baker. 2020. “Protein Sequence Optimization with a Pairwise Decomposable Penalty for Buried Unsatisfied Hydrogen Bonds.” Cold Spring Harbor Laboratory. doi.org/10.1101/2020.06.17.156646.
DiMaio, Frank, Andrew Leaver-Fay, Phil Bradley, David Baker, and Ingemar André. 2011. “Modeling Symmetric Macromolecular Structures in Rosetta3.” PloS One 6 (6): e20450.
Doyle, Lindsey, Jazmine Hallinan, Jill Bolduc, Fabio Parmeggiani, David Baker, Barry L. Stoddard, and Philip Bradley. 2015. “Rational Design of α-Helical Tandem Repeat Proteins with Closed Architectures.” Nature 528 (7583): 585-88.
Dyer, Kevin N., Michal Hammel, Robert P. Rambo, Susan E. Tsutakawa, Ivan Rodic, Scott Classen, John A. Tainer, and Greg L. Hura. 2014. “High-Throughput SAXS for the Characterization of Biomolecules in Solution: A Practical Approach.” Methods in Molecular Biology 1091: 245-58.
Erickson, J., and D. Kempf 1994. “Structure-Based Design of Symmetric Inhibitors of HIV-1 Protease.” Archives of Virology. Supplementum 9: 19-29.
Faiella, Marina, Concetta Andreozzi, Rafael Tones Martin de Rosales, Vincenzo Pavone, Ornella Maglio, Flavia Nastri, William F. DeGrado, and Angela Lombardi. 2009. “An Artificial Di-Iron Oxo-Protein with Phenol Oxidase Activity.” Nature Chemical Biology 5 (12): 882-84.
Fallas, Jorge A., George Ueda, William Sheffler, Vanessa Nguyen, Dan E. McNamara, Banumathi Sankaran, Jose Henrique Pereira, et al. 2017. “Computational Design of Self-Assembling Cyclic Protein Homo-Oligomers.” Nature Chemistry 9 (4): 353-60.
Foight, Glenna Wink, Zhizhi Wang, Cindy T. Wei, Per Jr Greisen, Katrina M. Warner, Daniel Cunningham-Bryant, Keunwan Park, et al. 2019. “Multi-Input Chemical Control of Protein Dimerization for Programming Graded Cellular Responses.” Nature Biotechnology 37 (10): 1209-16.
“FoXS Server: Fast X-Ray Scattering.” n.d. Accessed Sep. 17, 2020.

modbase.compbio.ucsf.edu/foxs/.
Gibney, B. R., S. E. Mulholland, F. Rabanal, and P. L. Dutton. 1996. “Ferredoxin and Ferredoxin-Heme Maquettes.” Proceedings of the National Academy of Sciences of the United States of America 93 (26): 15041-46.
Greenfield, Norma J. 2006. “Using Circular Dichroism Spectra to Estimate Protein Secondary Structure.” Nature Protocols 1 (6): 2876-90.
Hauser, Kevin, Yiqing He, Miguel Garcia-Diaz, Carlos Simmerling, and Evangelos Coutsias. 2017. “Characterization of Biomolecular Helices and Their Complementarity Using Geometric Analysis.” Journal of Chemical Information and Modeling 57 (4): 864-74.
Hura, Greg L., Helen Budworth, Kevin N. Dyer, Robert P. Rambo, Michal Hammel, Cynthia T. McMurray, and John A. Tainer. 2013. “Comprehensive Macromolecular Conformations Mapped by Quantitative SAXS Analyses.” Nature Methods 10 (6): 453-54.
Hura, Greg L., Angeli L. Menon, Michal Hammel, Robert P. Rambo, Farris L. Poole 2nd, Susan E. Tsutakawa, Francis E. Jenney Jr, et al. 2009. “Robust, High-Throughput Solution Structural Analyses by Small Angle X-Ray Scattering (SAXS).” Nature Methods 6 (8): 606-12.
Leman, Julia Koehler, Brian D. Weitzner, Steven M. Lewis, Jared Adolf-Bryfogle, Nawsad Alam, Rebecca F. Alford, Melanie Aprahamian, et al. 2020. “Macromolecular Modeling and Design in Rosetta: Recent Methods and Frameworks.” Nature Methods 17 (7): 665-80.
Maguire, Jack B., Hugh K. Haddox, Devin Strickland, Samer F. Halabiya, Brian Coventry, Jermel R. Griffin, Surya V. S. R. K. Pulavarti, et al. 2021. “Perturbing the Energy Landscape for Improved Packing during Computational Protein Design.” Proteins 89 (4): 436-49.
Norn, Christoffer, Basile I. M. Wicky, David Juergens, Sirui Liu, David Kim, Doug Tischer, Brian Koepnick, et al. 2021. “Protein Sequence Design by Conformational Landscape Optimization.” Proceedings of the National Academy of Sciences of the United States of America 118 (11). doi.org/10.1073/pnas.2017228118.
Oie, Tetsuro, Gerald M. Maggiora, and Ralph E. Christoffersen. 2009. “Structural Characterization of a Special-Pair Chlorophyll Dimer Model of P700.” International Journal of Quantum Chemistry. doi.org/10.1002/qua.560220716.
Park, Jooyoung, Brinda Selvaraj, Andrew C. McShan, Scott E. Boyken, Kathy Y. Wei, Gustav Oberdorfer, William DeGrado, et al. 2019. “De Novo Design of a Homo-Trimeric Amantadine-Binding Protein,” December. doi.org/10.7554/eLife.47839.
Pyles, Harley, Shuai Zhang, James J. De Yoreo, and David Baker. 2019. “Controlling Protein Assembly on Inorganic Crystals through Designed Protein Interfaces.” Nature 571 (7764): 251-56.
“SAXS FrameSlice.” n.d. Accessed Sep. 17, 2020. sibyls.a1s.1b1.gov/ran. Schneidman-Duhovny, Dina, Michal Hammel, John A. Tainer, and Andrej Sali. 2013. “Accurate SAXS Profile Computation and Its Assessment by Contrast Variation Experiments.” Biophysical Journal 105 (4): 962-74.
Sharp, P. M., and W. H. Li. 1987. “The Codon Adaptation Index—a Measure of Directional Synonymous Codon Usage Bias, and Its Potential Applications.” Nucleic Acids Research 15 (3): 1281-95.
Ueda, George, Aleksandar Antanasijevic, Jorge A. Fallas, William Sheffler, Jeffrey Copps, Daniel Ellis, Geoffrey B. Hutchinson, et al. 2020. “Tailored Design of Protein Nanoparticle Scaffolds for Multivalent Presentation of Viral Glycoprotein Antigens.” eLife 9 (August). doi.org/10.7554/eLife.57659.

LENGTHY TABLES
The patent application contains a lengthy table section. A copy of the table is available in electronic form from the USPTO web site (https://seqdata.uspto.gov/?pageRequest=docDetail&DocID=US20230114825A1). An electronic copy of the table will also be available from the USPTO upon request and payment of the fee set forth in 37 CFR 1.19(b)(3).

We claim:

1. A polypeptide comprising an amino acid sequence at least 50% identical to the amino acid sequence selected from the group consisting of SEQ ID NOS:1-3297, not including any functional domains added fused to the polypeptides (whether N-terminal, C-terminal, or internal), and wherein the 1, 2, 3, 4, or 5 N-terminal and/or C-terminal amino acid residues may be present or absent and when absent are not considered in determining the percent identity.

2. The polypeptide of claim 1, comprising an amino acid sequence at least 75% identical to the amino acid sequence selected from the group consisting of SEQ ID NOS:1-3297, not including any functional domains added fused to the polypeptides (whether N-terminal, C-terminal, or internal), and wherein the 1, 2, 3, 4, or 5 N-terminal and/or C-terminal amino acid residues may be present or absent and when absent are not considered in determining the percent identity.

3. The polypeptide of claim 1, comprising an amino acid sequence at least 90% identical to the amino acid sequence selected from the group consisting of SEQ ID NOS:1-3297, not including any functional domains added fused to the polypeptides (whether N-terminal, C-terminal, or internal), and wherein the 1, 2, 3, 4, or 5 N-terminal and/or C-terminal amino acid residues may be present or absent and when absent are not considered in determining the percent identity.

4. The polypeptide of claim 1, comprising the amino acid sequence selected from the group consisting of SEQ ID NOS:1-3297, not including any functional domains added fused to the polypeptides (whether N-terminal, C-terminal, or internal), and wherein the 1, 2, 3, 4, or 5 N-terminal and/or C-terminal amino acid residues may be present or absent and when absent are not considered in determining the percent identity.

5. The polypeptide of claim 1, wherein all residues are included when determining the percent identity relative to the reference polypeptide.

6. The polypeptide of claim 1, wherein amino acid substitutions relative to the reference polypeptide are conservative amino acid substitutions. 7 The polypeptide of claim 1, further comprising one or more functional domains.

8. A homodimer of the polypeptide of claim 1.

9. The homodimer of claim 8, comprising a ligand bound in a pocket of the homodimer.

10. The homodimer of claim 9, wherein the ligand comprises a C2 symmetric compound.

11. A composition comprising 2 or more different polypeptides or homodimers of claim 1.

12. A nucleic acid encoding the polypeptide of claim 1.

13. An expression vector comprising the nucleic acid of claim 12 operatively linked to a suitable control sequence.

14. A host cell comprising the expression vector of claim 13.

15. A pharmaceutical composition comprising:
(a) the polypeptide claim 1; and
(b) a pharmaceutically acceptable carrier.

16. A method for using the polypeptide of claim 1 for any suitable use as disclosed herein.
